(19) United States
(12) Patent Application Publication
(10) Pub. No.: US 2026/0083399 A1
(43) Pub. Date: Mar. 26, 2026

(54) SENSOR AND DETECTION DEVICE

(71) Applicant: Japan Display Inc., Tokyo (JP)

(72) Inventor: Mamoru DOUYOU, Tokyo (JP)

(73) Assignee: Japan Display Inc., Tokyo (JP)

(21) Appl. No.: 19/333,442

(22) Filed: Sep. 19, 2025

(30) Foreign Application Priority Data
Sep. 20, 2024 (JP) 2024-163844

(51) Int. Cl. A61B 5/00

(52) U.S. Cl. CPC A61B 5/6826; A61B 5/0059; A61B 2562/0233

(57) ABSTRACT

According to one embodiment, a sensor includes a first insulating substrate including a first island portion and a second island portion arranged along a first direction, and a band portion disposed between the first island portion and the second island portion and connecting the first island portion and the second island portion with each other, a photodiode disposed above the first insulating substrate, a sealing layer sealing the photodiode, and a reflective member disposed above the sealing layer, and the reflective member reflects light passing through the first insulating substrate and made incident on the reflective member.

### CROSS-REFERENCE TO RELATED APPLICATIONS

This application is based upon and claims the benefit of priority from Japanese Patent Application No. 2024-163844, filed Sep. 20, 2024, the entire contents of which are incorporated herein by reference.

### FIELD

Embodiments described herein relate generally to a sensor and detection device.

### BACKGROUND

Optical sensors which can detect fingerprint patterns and vein patterns are known. Such optical sensors are in some cases incorporated into a ring-shaped housing to form a ring-shaped detection device. As to such sensors, there is a need for a sensor which can improve the light collection efficiency to the light receiving portion.

### BRIEF DESCRIPTION OF THE DRAWINGS

FIG. 1 is a diagram schematically showing a configuration example of a detection device of an embodiment when a finger is inserted therein, as viewed from a side of a housing thereof.

FIG. 2 is a cross-sectional view schematically showing the detection device taken along the line II-II in FIG. 1.

FIG. 3 is a plan view showing a configuration example of the detection device of this embodiment.

FIG. 4 a cross-sectional view schematically showing the detection device taken along the line IV-IV in FIG. 3.

FIG. 5 is a plan view showing a configuration example of the detection device shown in FIG. 2.

FIG. 6 is a plan view showing another configuration example of the detection device shown in FIG. 3.

FIG. 7 is a cross-sectional view schematically showing the detection device taken along the line VII-VII in FIG. 6.

### DETAILED DESCRIPTION

In general, according to an embodiment, a sensor comprises a first insulating substrate including a first island portion and a second island portion arranged along a first direction, and a band portion disposed between the first island portion and the second island portion and connecting the first island portion and the second island portion with each other, a photodiode disposed above the first insulating substrate, a sealing layer sealing the photodiode, and a reflective member disposed above the sealing layer, and the reflective member reflects light passing through the first insulating substrate and made incident on the reflective member.

According to another embodiment, a detection device comprises a sensor, and a ring-shaped housing, and the sensor comprises a first insulating substrate including a first island portion and a second island portion arranged in a first direction, and a band portion disposed between the first island portion and the second island portion and connecting the first island portion and the second island portion with each other, a photodiode disposed above the first insulating substrate, a sealing layer sealing the photodiode, a reflective member disposed above the sealing layer, the reflective member reflects light passing through the first insulating substrate and made incident on the reflective member, and the sensor is disposed inside the housing.

With configurations such as described above, it is possible to provide a sensor which can improve the light collection efficiency to the light receiving portion.

The embodiments will now be described hereinafter with reference to the accompanying drawings. Note that the disclosure is merely an example, and proper changes within the spirit of the invention, which are easily conceivable by a skilled person, are included in the scope of the invention as a matter of course. In addition, as to the drawings, in order to make the description clearer, the widths, thicknesses, shapes, etc., of the respective parts are schematically illustrated in the drawings, compared to the actual modes. However, the schematic illustration is merely an example, and adds no restrictions to the interpretation of the invention. Besides, in the specification and drawings, the same or similar elements as or to those described in connection with preceding drawings or those exhibiting similar functions are denoted by like reference numerals, and a detailed description thereof is omitted unless otherwise necessary.

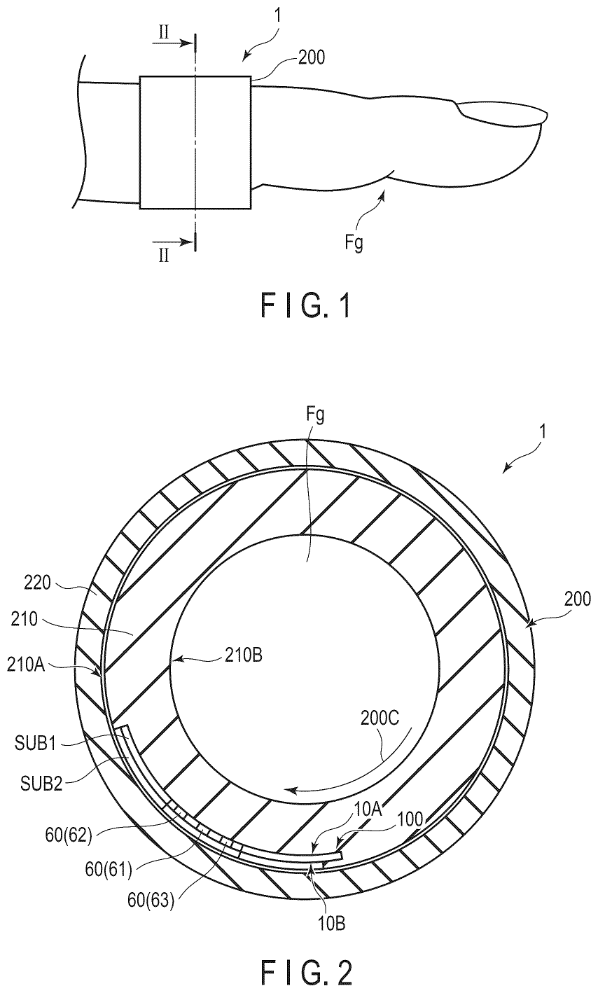

FIG. 1 is a schematic diagram showing an example of a detection device 1 of the present embodiment, when a finger Fg is inserted therein, as viewed from a side of a housing 200.

The detection device 1 shown in FIG. 1 is a ring-shaped device that can be attached to and detached from an object to be detected, such as a human body. In the example shown in FIG. 1, the detection device 1 is attached to the finger Fg of the human body, but not only that, it may as well be attached to some other part of the human body, such as the wrist or leg. The finger Fg includes a thumb, index finger, middle finger, ring finger, and little finger. The detection device 1 can detect information of the body part of the object to be detected, that is, biological information, from the finger Fg or other parts to which the detection device 1 is attached.

FIG. 2 is a cross-sectional view schematically showing the detection device 1 taken along the line II-II in FIG. 1.

As shown in FIG. 2, the detection device 1 comprises a sensor 100 and a ring-shaped housing 200. In this embodiment, the sensor 100 is an optical sensor that receives light and outputs an electrical signal corresponding to the received light. The detection device 1 further contains a battery (not shown) inside the housing 200. The detection device 1 operates using power from the battery.

In the example shown in FIG. 2, the housing 200 comprises a first housing 210 and a second housing 220. The first housing 210 has a ring shape and includes an outer circumferential surface 210A and an inner circumferential surface 210B. The first housing 210 is brought into contact with the object to be detected, such as finger Fg, via its inner circumferential surface 210B. The second housing 220 covers the outer circumferential surface 210A of the first housing 210. Between the first housing 210 and the second housing 220, there is an air layer. The sensor 100 is disposed inside the housing 200. In the example shown in FIG. 2, the sensor 100 is housed in the first housing 210, and the surface of the sensor 100, which faces the second housing 220 is in contact with the air layer.

The first housing 210 is formed, for example, from such a material as light-transmissive synthetic resin or silicon. The second housing 220 is formed, for example, from such a material as metal or non-transmissive synthetic resin.

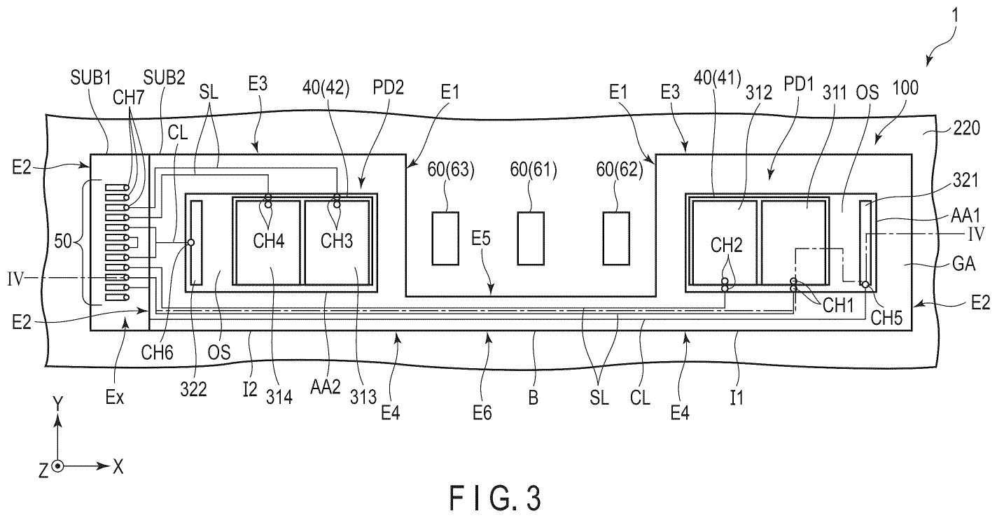

FIG. 3 is a plan view showing a configuration example of the detection device 1 of this embodiment.

In one example, a first direction X, a second direction Y, and a third direction Z are set orthogonal with each other, but may intersect at angles other than 90 degrees. The first direction X and the second direction Y correspond to directions parallel to the main surface of the substrate which constitutes the sensor 100, and the third direction Z corresponds to the thickness direction of the sensor 100. In this specification, the direction from the first substrate SUB1 toward the second substrate SUB2 is referred to as the “upper side” (or simply ‘up’), and the direction from the second substrate SUB2 toward the first substrate SUB1 is referred to as the “lower side” (or simply “down”). With such expressions as “the second member above the first member” and “the second member below the first member,” the second member may be in contact with the first member or may be spaced apart from the first member. Further, supposing that there is an observation position where the sensor 100 is observed from the tip side of the arrow indicating the third direction Z, viewing toward the X-Y plane defined by the first direction X and the second direction Y from this observation position, is referred to as plan view. Note that the first direction X shown in FIG. 3 coincides with an inner circumferential direction 200C of the housing 200 shown in FIG. 2.

As shown in FIG. 3, the detection device 1 comprises a sensor 100 and a ring-shaped housing 200. The housing 200 includes a first housing 210 and a second housing 220. Note that in FIG. 3, the first housing 210 is omitted from the illustration.

The sensor 100 comprises a first substrate SUB1 and a second substrate SUB2. In FIG. 3, the first substrate SUB1 and the second substrate SUB2 are shown through the second housing 220.

The first substrate SUB1 and the second substrate SUB2 are formed as flat plates parallel to the X-Y plane. The first substrate SUB1 and the second substrate SUB2 may be bent in the third direction Z, for example.

The first substrate SUB1 includes a first island portion I1, a second island portion I2, and a band portion B disposed between the first island portion I1 and the second island portion I2 and connecting the first island portion I1 and the second island portion I2 to each other.

In the example shown in FIG. 3, each of the first island portion I1 and the second island portion I2 of the first substrate SUB1 has an approximately rectangular shape. Each of the first island portion I1 and the second island portion I2 of the first substrate SUB1 has edges E1 and E2 which extend along the second direction Y and face each other along the first direction X. The edge E1 of the first island portion I1 faces the edge E1 of the second island portion I2 along the first direction X.

In the example shown in FIG. 3, each of the first island portion I1 and the second island portion I2 of the first substrate SUB1 has edges E3 and E4 which extend along the first direction X and face each other along the second direction Y.

In the example shown in FIG. 3, the band portion B of the first substrate SUB1 has edges E5 and E6 which extends along the first direction X and face each other along the second direction Y. The edge E5 faces the light source 60, which will be described later, along the second direction Y.

The band portion B of the first substrate SUB1 connects the first island portion I1 and the second island portion I2 of the first substrate SUB1 with each other. In the example shown in FIG. 3, one end of the band portion B is connected to the edge E1 of the first island portion I1. The other end of the band portion B is connected to the edge E1 of the second island portion I2. More specifically, the connection portion between one end of the band portion B and the first island portion I1 is located close to an edge E4 side in the second direction Y. Similarly, the connection portion between the other end of the band portion B and the second island portion I2 is located close to the edge E4 side in the second direction Y. In the example shown in FIG. 3, the edge E6 is continuously connected to the edge E4 of the first island portion I1 and the edge E4 of the second island portion I2.

The second substrate SUB2 includes the first island portion I1, the second island portion I2, and a band portion B disposed between the first island portion I1 and the second island portion I2 and connecting the first island portion I1 and the second island portion I2 with each other.

In the example shown in FIG. 3, each of the first island portion I1 and the second island portion I2 of the second substrate SUB2 has an approximately rectangular shape. Each of the first island portion I1 and the second island portion I2 of the second substrate SUB2 has edges E1 and E2 which extend along the second direction Y and face each other along the first direction X. The edge E1 of the first island portion I1 faces the edge E1 of the second island portion I2 along the first direction X.

Further, in the example shown in FIG. 3, each of the first island portion I1 and the second island portion I2 of the second substrate SUB2 has edges E3 and E4 which extend along the first direction X and face each other along the second direction Y.

In the example shown in FIG. 3, the band portion B of the second substrate SUB2 has edges E5 and E6 which extend along the first direction X and face each other along the second direction Y. The edge E5 faces the light source 60, which will be described later, along the second direction Y.

The band portion B of the second substrate SUB2 connects the first island portion I1 and the second island portion I2 of the first substrate SUB1 with each other. In the example shown in FIG. 3, one end of the band portion B is connected to the edge E1 of the first island portion I1. The other end of the band portion B is connected to the edge E1 of the second island portion I2. More specifically, the connection portion between the one end of the band portion B and the first island portion I1 is located close to the edge E4 side along the second direction Y. Similarly, the connection portion between the other end of the band portion B and the second island portion I2 is located close to the edge E4 side along the second direction Y. In the example shown in FIG. 3, the edge E6 is continuously connected to the edge E4 of the first island portion I1 and the edge E4 of the second island portion I2.

The first substrate SUB1 and the second substrate SUB2 overlap each other in plan view. The edges E1 of the first substrate SUB1 and the second substrate SUB2 overlap each other in plan view. The edges E3 of the first substrate SUB1 and the second substrate SUB2 overlap each other in plan view. The edges E4 of the first substrate SUB1 and the second substrate SUB2 overlap each other in plan view. The edges E5 of the first substrate SUB1 and the second substrate SUB2 overlap each other in plan view. The edges E6 of the first substrate SUB1 and the second substrate SUB2 overlap each other in plan view.

The second island portion I2 of the first substrate SUB1 has an extending portion Ex extending in the first direction X from the edge E2 of the second substrate SUB2 in plan view. The extending portion Ex does not overlap the second substrate SUB2 in plan view. That is, the edges E2 of the second island portion I2 of the first substrate SUB1 and the second substrate SUB2 do not overlap each other in plan view.

In other words, the sensor 100, in plan view, includes a first island portion I1, a second island portion I2, and a band portion B disposed between the first island portion I1 and the second island portion I2, and connecting the first island portion I1 and the second island portion I2 with each other, in an area where the first substrate SUB1 and the second substrate SUB2 overlap each other. In the example shown in FIG. 3, each of the first island portion I1 and the second island portion I2 of the sensor 100 has edges E1 and E2 which extend along the second direction Y and face each other along the first direction X. Further, each of the first island portion I1 and the second island portion I2 of the sensor 100 has edges E3 and E4 which extend along the first direction X and face each other along the second direction Y. The band portion B of the sensor 100 has edges E5 and E6 which extend along the first direction X and face each other along the second direction Y.

In the example shown in FIG. 3, the sensor 100 has two detection areas AA1 and AA2 and a peripheral area GA in the area where the first substrate SUB1 and the second substrate SUB2 overlap each other. The detection area AA1 overlaps the first island portion I1 in plan view, and the detection area AA2 overlaps the second island portion I2 in plan view. The peripheral area GA surrounds detection the detection areas AA1 and AA2. Further, the peripheral area GA overlaps the band portion B in plan view.

The detection area AA1 comprises a first photodiode PD1, and the detection area AA2 comprises a second photodiode PD2. Note that in the following descriptions, the first photodiode PD1 and the second photodiode PD2 may be collectively referred to as photodiodes PD in some cases.

The first photodiode PD1 and the second photodiode PD2 are the light-receiving portions of the sensor 100. Each of the first photodiode PD1 and the second photodiode PD2 receives light and outputs an electrical signal corresponding to the received light. The first photodiode PD1 and the second photodiode PD2 are, for example, organic photodiodes (OPDs) formed by using organic semiconductors.

Each of the first photodiode PD1 and the second photodiode PD2 includes a semiconductor layer, a first electrode 31, and a second electrode 32. In the example shown in FIG. 3, the first photodiode PD1 includes an organic semiconductor layer OS, first electrodes 311 and 312, and a second electrode 321. The first electrodes 312, 311, and the second electrodes 321 are arranged in this order along the first direction X.

The organic semiconductor layer OS of the first photodiode PD1 overlaps the detection area AA1 in plan view. Further, the organic semiconductor layer OS of the first photodiode PD1 overlaps the first electrodes 311, 312, and the second electrode 321 in plan view, and is provided across the first electrodes 311, 312, and the second electrode 321.

In the example shown in FIG. 3, the second photodiode PD2 includes an organic semiconductor layer OS, first electrodes 313 and 314, and a second electrode 322. The second electrode 322 and the first electrodes 314 and 313 are arranged in this order along the first direction X.

The organic semiconductor layer OS of the second photodiode PD2 overlaps the detection area AA2 in plan view. Further, the organic semiconductor layer OS of the second photodiode PD2 overlaps the first electrodes 313, 314 and the second electrode 322 in plan view, and is disposed across the first electrodes 313, 314 and the second electrode 322.

The sensor 100 further comprises a reflective member 40. The reflective member 40 overlaps the first electrode 31 in plan view.

In the example shown in FIG. 3, the sensor 100 comprises a reflective member 41 overlapping the first island portion I1 and a reflective member 42 overlapping on the second island portion I2 in plan view. The reflective member 41 overlaps the first electrodes 311 and 312 in plan view. In the example shown in FIG. 3, the reflective member 41 does not overlap the peripheral area GA in plan view, but the configuration is not limited to this. For example, the reflective member 41 may as well overlap the peripheral area GA.

The reflective member 42 overlaps the first electrodes 313 and 314 in plan view. In the example shown in FIG. 3, the reflective member 42 does not overlap the peripheral area GA in plan view, but the configuration is not limited to this. For example, the reflective member 42 may as well overlap the peripheral area GA.

FIG. 3 illustrates an example in which the sensor 100 has two reflective members 41 and 42, but the configuration is not limited to this. For example, the sensor 100 may have a single reflective member 40, and the single reflective member 40 may overlap the detection areas AA1, AA2, and the peripheral area GA in plan view, and overlap the first electrodes 311, 312, 313, and 314.

The sensor 100 further includes a plurality of terminals 50. These terminals 50 are provided on the extending portion Ex of the first substrate SUB1. In the example shown in FIG. 3, the terminals 50 are arranged along the second direction Y. Each of the terminals 50 is connected to a control circuit (not shown).

The first electrodes 31 are electrically connected to signal lines SL, respectively. In the example shown in FIG. 3, the first electrodes 311, 312, 313, and 314 are respectively connected to the signal lines SL via respective contact holes (CH1, CH2, CH3, CH4) formed in an insulating layer 13, which will be described later.

The signal line SL connected to the first electrode 311 extends in the second direction Y from the connection point with the first electrode 311 (contact hole CH1), bends in the first direction X, and extends in the first direction X. The signal line SL connected to the first electrode 312 extends in the second direction Y from the connection point with the first electrode 312 (contact hole CH2), bends in the first direction X, and extends in the first direction X. The signal lines SL respectively connected to the first electrodes 311 and 312 each overlap the band portion B in plan view.

The signal line SL connected to the first electrode 313 extends in the second direction Y from the connection point with the first electrode 313 (contact hole CH3), bends in the first direction X, and extends in the first direction X. The signal line SL connected to the first electrode 314 extends in the second direction Y from the connection point with the first electrode 314 (contact hole CH4), bends in the first direction X, and extends in the first direction X. The signal lines SL respectively connected to the first electrodes 313 and 314 each do not overlap the band portion B in plan view.

Each signal line SL is connected to one of the terminals 50. That is, each of the first electrodes 311, 312, 313, and 314 is connected to the control circuit via the respective signal line SL and terminal 50.

The second electrodes 32 are electrically connected to the power supply line CL. In the example shown in FIG. 3, the second electrodes 321 and 322 are each connected to the power supply line CL via respective contact holes CH5 and CH6 formed in the insulating layer 13, which will be described later.

The power supply line CL connected to the second electrode 321 extends in the second direction Y from the connection point with the second electrode 321 (contact hole CH5), bends in the first direction X, and extends in the first direction X. The power supply line CL connected to the second electrode 321 overlaps the band portion B in plan view.

The power supply line CL connected to the second electrode 322 extends in the second direction Y from the connection point with the second electrode 322 (contact hole CH6). The power supply line CL connected to the second electrode 322 does not overlap the band portion B in plan view.

The power supply line CL is connected to one of the terminals 50. That is, the second electrodes 321 and 321 are each connected to the control circuit via the power supply line CL and the respective terminal 50.

The signal line SL and the power supply line CL overlap the peripheral area GA in plan view. The signal line SL and the power supply line CL are provided in the same layer.

The control circuit supplies control signals to the first photodiode PD1 and the second photodiode PD2 so as to control the detection operation. Each of the first photodiode PD1 and the second photodiode PD2 outputs an electrical signal corresponding to the received light as a detection signal to the control circuit. The detection device 1 detects information related to the object to be detected based on the detection signals.

The sensor 100 further comprises light sources 60. In the example shown in FIG. 3, the sensor 100 includes three light sources 60, namely, a first light source 61, a second light source 62, and a third light source 63.

The first light source 61 is disposed between the first island portion I1 and the second island portion I2 in a plan view. The second light source 62 is disposed between the first island portion I1 and the first light source 61 in the first direction X. The third light source 63 is disposed between the second island portion I2 and the first light source 61 in the first direction X.

The light sources 60 do not overlap the first substrate SUB1 and the second substrate SUB2 in plan view. The light sources 60 are provided, for example, in the first housing 210. Although not shown, the sensor 100 may further comprise a third substrate SUB3 that overlaps the first substrate SUB1 and the second substrate SUB2 in plan view, and the light sources 60 may be disposed on the third substrate SUB3.

For the light sources 60, for example, inorganic light emitting diode (LED) or organic EL (organic light emitting diode (OLED) may be used. For example, the first light source 61 emits infrared light or red light, and the second light source 62 and the third light source 63 emit green light.

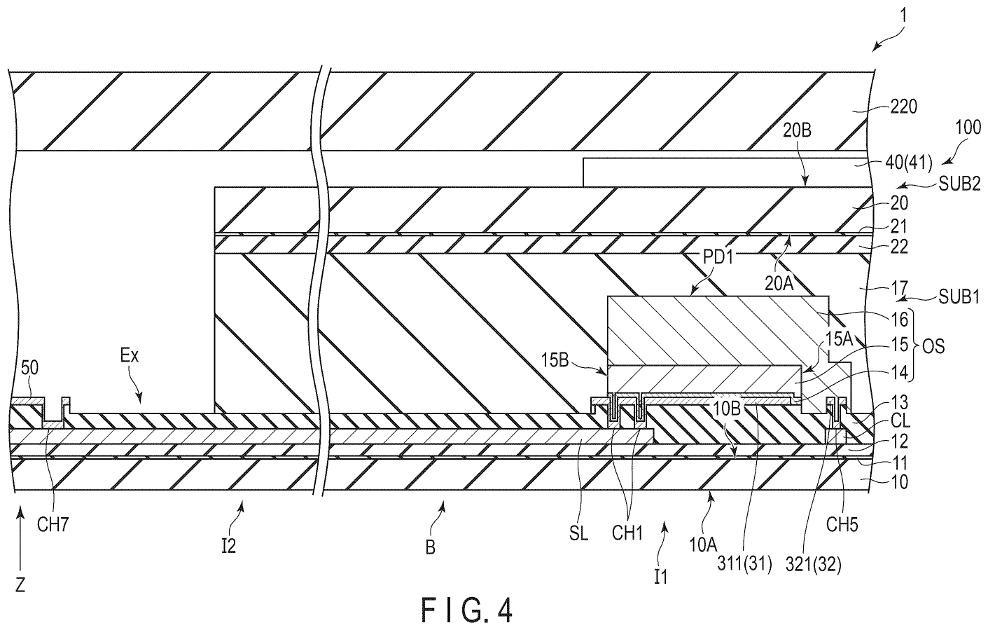

FIG. 4 is a cross-sectional view schematically showing the detection device 1 taken along the line IV-IV in FIG. 3. Note that in FIG. 4, the second housing 220, the first island portion I1, the band portion B, and the extending portion Ex of the sensor 100 are mainly shown, and other elements are omitted.

The first substrate SUB1 comprises an insulating substrate 10, a protective layer 11, a buffer layer 12, an insulating layer 13, a first photodiode PD1, a second photodiode PD2, a sealing layer 17, signal lines SL, a power supply line CL, and terminals 50.

The insulating substrate 10 has a main surface (lower surface) 10A and a main surface (upper surface) 10B on an opposite side to the main surface 10A. The protective layer 11 covers the main surface 10B over the first island portion I1, the second island portion I2, and the band portion B. The buffer layer 12 covers the protective layer 11. Each of the signal lines SL and the power supply line CL is disposed on the buffer layer 12.

The insulating layer 13 covers the buffer layer 12, the signal lines SL, and the power supply line CL. The first photodiode PD1 is disposed on the insulating layer 13 which overlaps the first island portion I1 in plan view. Although not shown, the second photodiode PD2 is disposed on the insulating layer 13 which overlaps the second island portion I2 in plan view.

The first photodiode PD1 comprises first electrodes 311 and 312, a second electrode 321, and an organic semiconductor layer OS. Note that in FIG. 4, the first electrode 312 is omitted from the illustration. Although not shown, the second photodiode PD2 comprises first electrodes 313 and 314, a second electrode 322, and an organic semiconductor layer OS.

The first electrodes 311, 312 and the second electrode 321 are disposed on the insulating layer 13. Although not shown, the first electrodes 313, 314 and the second electrode 322 as well are disposed on the insulating layer 13. The terminals 50 are disposed on the insulating layer 13, which overlaps the extending portion Ex in plan view.

The first electrode 311 is electrically connected to the respective signal line SL through the contact hole (CH1) formed in the insulating layer 13.

Although not shown, the first electrodes 312, 313, and 314 as well are each electrically connected to the respective signal line SL via respective contact holes (CH2, CH3, and CH4) formed in the insulating layer 13.

The second electrode 321 is electrically connected to the power supply line CL via a contact hole (CH5) formed in the insulating layer 13. Although not shown, the second electrode 322 as well is electrically connected to the power supply line CL via a contact hole (CH6) formed in the insulating layer 13.

The respective terminal 50 is electrically connected to the signal line SL via a contact hole (CH7) formed in the insulating layer 13.

The organic semiconductor layer OS, which overlaps the first island portion I1 in plan view, covers the first electrodes 311 and 312 and the second electrode 321. Although not shown, the organic semiconductor layer OS, which overlaps the second island portion I2 in plan view, covers the first electrodes 313 and 314 and the second electrode 322. The organic semiconductor layer OS includes an electron injection layer 14, an active layer 15, and a hole injection layer 16, as shown in FIG. 4.

The electron injection layer 14, which overlaps the first island portion I1 in plan view, continuously covers the first electrodes 311 and 312. The electron injection layer 14 is in contact with the first electrodes 311 and 312. Although not shown, the electron injection layer 14, which overlaps the second island portion I2 in plan view, continuously covers the first electrodes 313 and 314 and is in contact with the first electrodes 313 and 314. Note that, as shown in FIG. 4, a part of the first electrode 31 may be exposed from the electron injection layer 14.

The active layer 15 covers the upper surface of the electron injection layer 14 and is in contact with the electron injection layer 14. The hole injection layer 16 continuously covers the upper surface of the active layer 15, the side surface 15A which faces the second electrode 32, and the second electrode 32. The hole injection layer 16 is in contact with the active layer 15. Further, the hole injection layer 16 is in contact with the second electrode 32. As shown in FIG. 4, the side surface 15B of the active layer 15, which is on an opposite side to the side surface 15A, may be exposed from the hole injection layer 16.

The sealing layer 17 is disposed over the first island portion I1, the second island portion I2, and the band portion B. The sealing layer 17 covers the insulating layer 13, the hole injection layer 16, the side surface 15B of the active layer 15, and the first electrode 31 exposed from the active layer 15. With the thus sealing layer 17 formed, the photodiode PD is effectively sealed, thereby making it possible to suppress the entering of moisture from the upper surface side.

The sealing layer 17 is not disposed on the extending portion Ex. That is, in the extending portion Ex, the insulating layer 13, contact hole CH7, and terminal 50 are exposed from the sealing layer 17.

The second substrate SUB2 comprises an insulating substrate 20, a protective layer 21, a buffer layer 22, and reflective members 41 and 42. The insulating substrate 20 has a main surface (lower surface) 20A and a main surface (upper surface) 20B on an opposite side to the main surface 20A. The protective layer 21 covers the main surface 20A. The buffer layer 22 covers the protective layer 21. The protective layer 21 is adhered to the first substrate SUB1 by the sealing layer 17, for example. With this configuration, the first substrate SUB1 and the second substrate SUB2 are adhered to each other.

The reflective member 41 is provided on the main surface 20B. The reflective members 41 overlap the first electrodes 311 and 312 in plan view. Although not shown, the reflective member 42 as well is provided on the main surface 20B, and overlaps the first electrodes 313 and 314 in plan view.

The sensor 100 of this embodiment shown in FIGS. 3 and 4 is housed in the first housing 210 as shown in FIG. 2, and thus it is disposed inside the ring-shaped housing 200. The main surface 10A of the insulating substrate 10 faces the inner circumferential surface 210B of the first housing 210, and the main surface 10B of the insulating substrate 10 faces the second housing 220. The sensor 100 is curved along the outer circumferential surface 210A of the first housing 210.

The reflective members 41 and 42 are disposed between the insulating substrate 10 and the second housing 220, as shown in FIG. 4, and are disposed between the insulating substrate 20 and the second housing 220.

As shown in FIG. 4, between sensor 100 and second housing 220, there is an air layer. The surface of the reflective member 40, which faces the second housing 220 is in contact with the air layer.

The insulating substrates 10 and 20 are insulating substrates and have flexibility. The insulating substrates 10 and 20 are formed, for example, from film-like resin. The protective layers 11 and 21 are formed from an inorganic insulating material, such as a SiOx film. The buffer layers 12 and 22 are formed from an organic material. The insulating layer 13 may as well be an inorganic insulating film or an organic insulating film. Further, the insulating layer 13 may be a single layer or a multilayer film. The signal lines SL and the power supply line CL are formed, for example, from metal wiring lines.

The first electrode 31 and the second electrode 32 are formed, for example, from a conductive material having transparency, such as indium tin oxide (ITO) or indium zinc oxide (IZO).

The electron injection layer 14 is formed from a material having electron injection characteristics. As the electron injection layer 14, materials generally used as for electron injection layers can be employed. The hole injection layer 16 is formed from a material having hole injection characteristics. As the hole injection layer 16, materials generally used as for hole injection layers can be employed.

The active layer 15 is formed from a material whose properties (for example, voltage-current characteristics or resistance value) change in response to irradiated light. For example, the active layer 15 has a bulk heterostructure in which a p-type organic semiconductor and an n-type organic semiconductor such as an n-type fullerene derivative (PCBM), are mixedly present. Further, as the active layer 15, for example, a low-molecular-weight organic material such as fullerene (C60), phenyl C61 butyric acid methyl ester (PCB), copper phthalocyanine (CuPc), fluorinated copper phthalocyanine (F16CuPc), 5,6,11,12-tetraphenyltetracene (rubrene), or perylene derivative (PDI) can be used.

The active layer 15 can be formed using any of these low-molecular-weight organic materials via a vapor deposition process (dry process). In this case, the active layer 15 may be, for example, a stacked layered film of CuPC and F16CuPC, or a stacked layered film of Rubrene and C60. The active layer 15 may as well be formed using a coating process (wet process).

In this case, for the active layer 15, a material prepared by combining a low-molecular-weight organic material such as described above and a high-molecular-weight organic material are combined may be used. As the high-molecular-weight organic material, for example, poly(3-hexylthiophene) (P3HT), F8-alt-benzothiadiazole (F8BT) or the like may be used. The active layer 15 can be a film in a state where P3HT and PCBM are mixed, or a film in a state where F8BT and PDI are mixed.

The sealing layer 17 is a sealing adhesive layer formed from an inorganic film such as a silicon nitride film or an aluminum oxide film, or a resin film such as acrylic. The sealing layer 17 is not limited to a single layer, and may be a stacked layer structure of two or more layers in which the above-listed inorganic films and resin film are combined.

The reflective member 40 is a film which can reflect light entering the reflective member 40. The reflective member 40 reflects at least one of infrared light, red light, and green light. As the reflective member 40, a reflective sheet such as Enhanced Specular Reflector (ESR) Film (manufactured by 3M) may be used. Further, as the reflective member 40, a resin film such as PET may be used, which includes a metal layer of a high-reflectivity metal material such as aluminum, silver, or titanium formed on a surface opposing the photodiode PD. The metal layer may be formed, for example, by vapor-depositing the metal material onto the surface of the resin film.

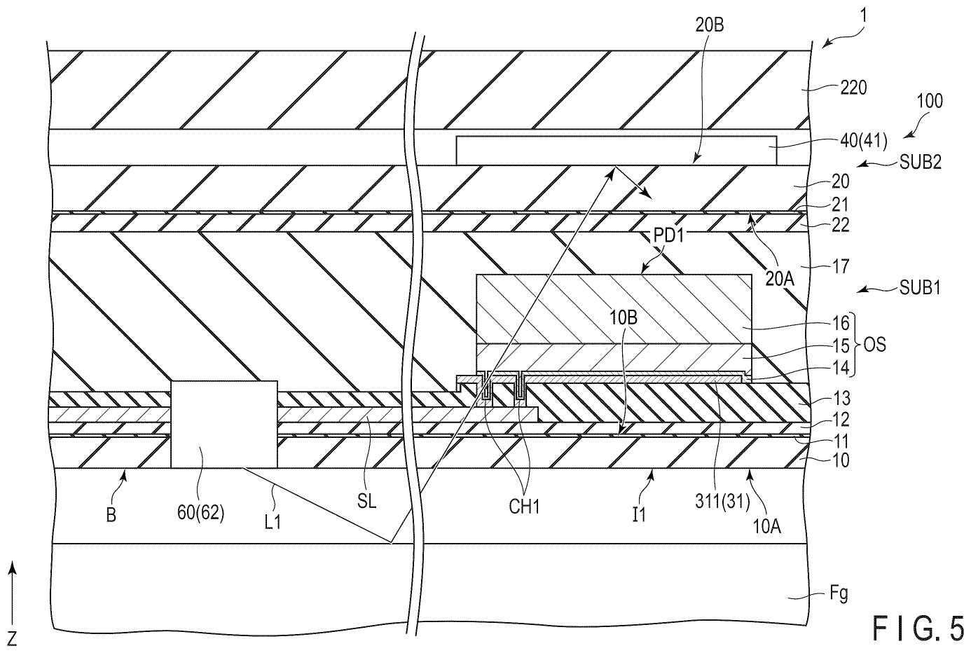

FIG. 5 is a cross-sectional view showing a configuration example of the detection device 1 shown in FIG. 2. Note that in FIG. 5, the second housing 220 and the sensor 100 of the detection device 1 are mainly illustrated and other elements are omitted. Further, FIG. 5 shows an example in which the finger Fg is placed inside the detection device 1 as the object to be detected. With reference to FIG. 5, the light emitted from the light sources 60 will be described.

The light sources 60 emit light L1 toward the object to be detected, such as the finger Fg placed inside the detection device 1. The light L1 emitted from the light sources 60 is either reflected off the surface or interior of the finger Fg or transmitted through finger Fg. FIG. 5 shows an example where the light L1 is reflected by the surface of the finger Fg. The light L1 that is reflected by the finger Fg or passes therethrough is transmitted through the insulating substrate 10 and enters at least one of the first photodiode PD1 and the second photodiode PD2. As a result, the voltage-current characteristics or resistance value of the active layer 15 changes, causing current to flow between the first electrode 31 and the second electrode 32 via the electron injection layer 14, the active layer 15, and the hole injection layer 16. The sensor 100 detects the current flowing between the first electrode 31 and the second electrode 32, thereby detecting biological information of the object to be detected.

Information related to the living body includes, for example, fingerprints, pulse waves of fingers or palms, heartbeat, vascular images, and blood oxygen saturation. Different information related to the living body can be detected depending on the type of the light L1 emitted from the light sources 60. For example, when the light L1 emitted from the light sources 60 is green light, the heartbeat or the like of the object can be detected. Further, when the light L1 emitted from the light sources 60 is infrared light or red light, blood oxygen saturation or the like can be detected. For example, in the case where the first light source 61 emits infrared light or red light, and the second light source 62 and the third light source 63 emit green light, as shown in FIG. 3, in order to detect the pulse rate of the object to be detected, it is sufficient to illuminate at least one of the second light source 62 and the third light source 63, and to detect the blood oxygen saturation, it is sufficient to illuminate only the first light source 61.

Part of the light L1 entering the photodiode PD passes through the photodiode PD and the second substrate SUB2 and reaches the main surface 20B, and then is made incident on the reflective member 40. In this embodiment, the sensor 100 can reflect the light L1 reaching the main surface 20B toward the photodiode PD by the reflective member 40. With this configuration, light leakage between the sensor 100 and the housing 200 can be prevented. As a result, compared to the case where the reflective member 40 is not provided, the light collection efficiency of the light L1 to the photodiode PD, which is the light receiving portion of the sensor 100, can be improved.

Thus, according to this embodiment, it is possible to provide a sensor 100 which can improve the light collection efficiency to the light receiving portion.

Next, another configuration example of this embodiment will be described.

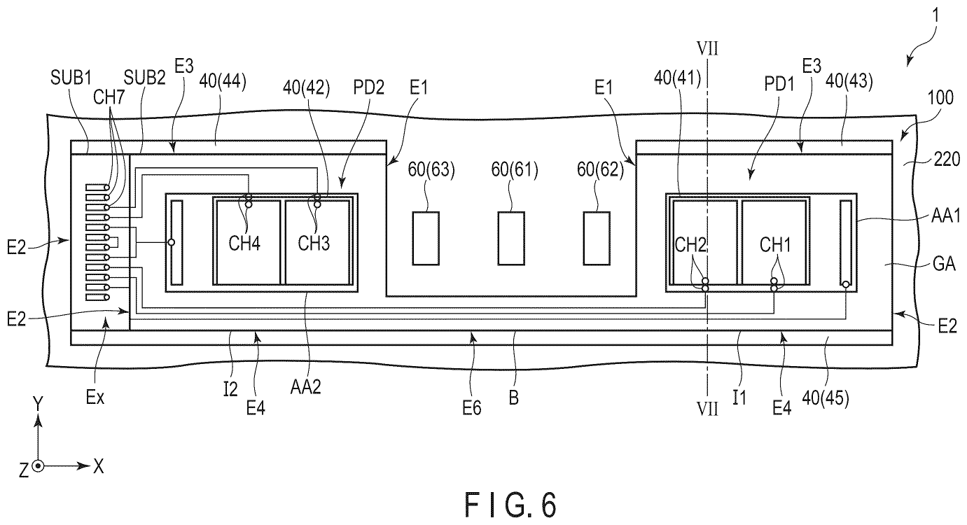

FIG. 6 is a plan view showing another configuration example of the detection device 1 shown in FIG. 3. The configuration example shown in FIG. 6 is different from the configuration example shown in FIG. 3 in that the sensor 100 further comprises a reflective member 40 on a side surface of the sensor 100.

In the example shown in FIG. 6, the sensor 100 further comprises a reflective member 43 along the edge E3 of the first island portion I1, and a reflective member 44 along the edge E3 of the second island portion I2. Further, the sensor 100 comprises a reflective member 45 along the edges E4 of the first island portion I1 and the second island portion I2, as well as along the edge E6 of the band portion.

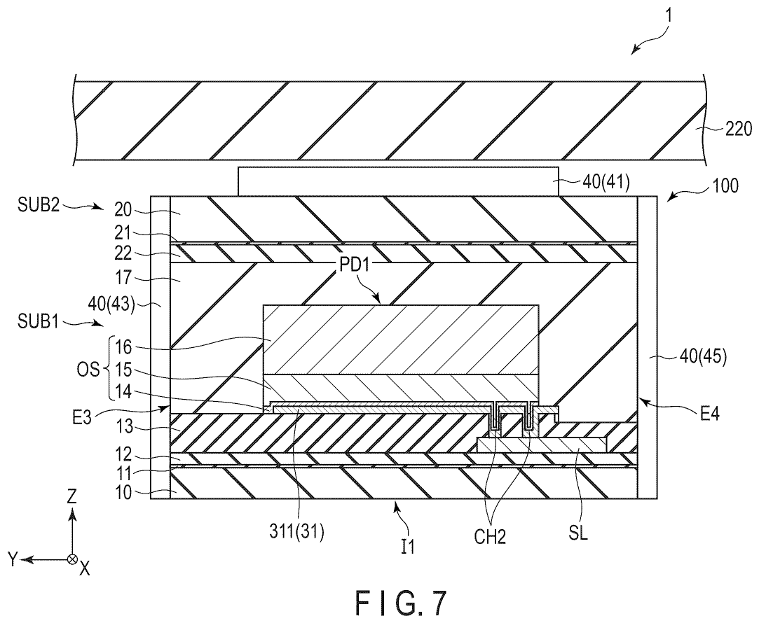

FIG. 7 is a cross-sectional view schematically showing the detection device 1 taken along the line VII-VII in FIG. 6. Note that in FIG. 7, the sensor 100 and the second housing 220 are mainly shown, and other elements are omitted. As shown in FIG. 7, the reflective member 43 is disposed on the side surface formed along the edge E3 of the first island portion I1. Although not shown, the reflective member 44 is disposed on the side surface along the edge E3 of the second island portion I2. The reflective member 45 is continuously disposed on the side surfaces along the edges E4 of the first island portion I1 and the second island portion I2, and the edge E6 of the band portion B.

In the example shown in FIG. 7, the reflective members 43, 44, and 45 are arranged over the side surfaces of the first substrate SUB1 and the second substrate SUB2, but the configuration is not limited to this. For example, the reflective members 43, 44, and 45 may be provided only on at least a part of the side surfaces of the first substrate SUB1 and the second substrate SUB2.

Here, note that depending on the incident angle of the light L1 emitted from the light sources 60 to the finger Fg, light leakage may occur from the side surface of the sensor 100, resulting in a decrease in the light collection efficiency of photodiode PD. However, the sensor 100 shown in FIGS. 6 and 7 can reflect the light L1 reaching the side surface of the sensor 100 toward the interior of the sensor 100 by the reflective member 40 provided on the side surface of the sensor 100. With this configuration, light leakage from the side surface of the sensor 100 can be prevented. Thus, compared to the case where the reflective member 40 is not provided on the side surface of the sensor 100, the light collection efficiency of the light L1 to the photodiode PD, which is the light receiving portion of the sensor 100, can be improved. As such, according to the configuration example shown in FIGS. 6 and 7, the light collection efficiency to the light receiving portion can be further improved. Furthermore, even in such a configuration example, advantageous effects similar to those presented in the configuration example shown in FIG. 3 can be obtained.

As described above, according to this embodiment, it is possible to provide a sensor which can improve the light collection efficiency to the light-receiving portion.

Based on the technical concept described above as embodiments of the invention, a person having ordinary skill in the art may achieve variations of various types with arbitral design changes; however, as long as they fall within the scope and spirit of the present invention, all of such products are encompassed by the scope of the present invention. A skilled person would conceive various changes and modifications of the present invention within the scope of the technical concept of the invention, and naturally, such changes and modifications are encompassed by the scope of the present invention. For example, if a skilled person adds/deletes/alters a structural element or design to/from/in the above-described embodiments, or adds/deletes/alters a step or a condition to/from/in the above-described embodiment, as long as they fall within the scope and spirit of the present invention, such addition, deletion, and altercation are encompassed by the scope of the present invention.

Furthermore, regarding the present embodiments, any advantage and effect those will be obvious from the description of the specification or arbitrarily conceived by a skilled person are naturally considered achievable by the present invention.

What is claimed is

1. A sensor comprising:
a first insulating substrate including a first island portion and a second island portion arranged along a first direction, and a band portion disposed between the first island portion and the second island portion and connecting the first island portion and the second island portion with each other;
a photodiode disposed above the first insulating substrate;
a sealing layer sealing the photodiode; and
a reflective member disposed above the sealing layer,
wherein
the reflective member reflects light passing through the first insulating substrate and made incident on the reflective member.

2. The sensor of claim 1, wherein
the reflective member reflects at least one of infrared light, red light, and green light.

3. The sensor of claim 1, wherein
the reflective member is a reflective sheet or a resin film having a metal layer on a surface thereof which faces the photodiode.

4. The sensor of claim 3, wherein
the metal layer is formed of aluminum.

5. The sensor of claim 1, wherein
the photodiode comprises:
a first electrode disposed above the first insulating substrate; and
a semiconductor layer disposed on the first electrode.

6. The sensor of claim 5, wherein
the reflective member overlaps the first electrode in plan view.

7. The sensor of claim 5, wherein
the semiconductor layer comprises
an electron injection layer in contact with the first electrode,
an active layer in contact with the electron injection layer, and
a hole injection layer in contact with the active layer.

8. The sensor of claim 7, further comprising:
a second electrode in contact with the hole injection layer.

9. The sensor of claim 8, further comprising:
an insulating layer disposed on the first insulating substrate.

10. The sensor of claim 9, wherein
the first electrode and the second electrode are disposed on the insulating layer.

11. The sensor of claim 1, further comprising:
a reflective member on a side surface.

12. The sensor of claim 1, further comprising:
a first light source disposed between the first island portion and the second island portion in plan view;
a second light source disposed between the first island portion and the first light source in the first direction; and
a third light source disposed between the second island portion and the first light source in the first direction.

13. The sensor of claim 12, wherein
the first light source emits infrared light or red light.

14. The sensor of claim 12, wherein
the second light source and the third light source emit green light.

15. A detection device comprising:
a sensor; and
a ring-shaped housing,
wherein the sensor comprises:
a first insulating substrate including a first island portion and a second island portion arranged in a first direction, and a band portion disposed between the first island portion and the second island portion and connecting the first island portion and the second island portion with each other;
a photodiode disposed above the first insulating substrate;
a sealing layer sealing the photodiode; and
a reflective member disposed above the sealing layer,
wherein
the reflective member reflects light passing through the first insulating substrate and made incident on the reflective member, and
the sensor is disposed inside the housing.

16. The detection device of claim 15, further comprising:
a first light source disposed between the first island portion and the second island portion in plan view;
a second light source disposed between the first island portion and the first light source in the first direction; and
a third light source disposed between the second island portion and the first light source in the first direction.

17. The detection device of claim 15, wherein
the housing comprises a first housing in a ring shape and a second housing covering an outer circumferential surface of the first housing, and
the sensor is housed in the first housing.

18. The detection device of claim 17, wherein
the reflection member is disposed between the first insulating substrate and the second housing.

19. The detection device of claim 16, wherein
the first light source emits infrared light or red light.

20. The detection device of claim 16, wherein
the second light source and the third light source emit green light.
